(19) United States
(12) Patent Application Publication
(10) Pub. No.: US 2012/0040886 A1
(43) Pub. Date: Feb. 16, 2012

(54) Analogues of Neuropeptide Y Having Proline Substitution At Position 34

(76) Inventor: Zheng Xin Dong, Holliston, MA (US)

(21) Appl. No.: 13/202,053

(22) PCT Filed: Feb. 19, 2010
(86) PCT No.: PCT/US10/00493

§ 371 Date: Oct. 26, 2011

Related U.S. Application Data
(60) Provisional application No. 61/208,153, filed on Feb. 20, 2009.

(51) Int. Cl. A61K 38/17; A61P 9/00; A61P 13/12; A61P 9/04; A61P 25/18; A61P 25/24; A61P 3/04; A61P 11/06; A61P 5/06; A61P 5/48; A61P 9/12; A61P 7/04; A61P 9/06; A61P 25/22; A61P 25/08; A61P 25/28; A61P 25/00; A61P 9/10; A61P 25/04; A61P 1/00; A61P 3/00; A61P 15/00; A61P 29/00; A61P 11/08; A61P 35/00; C07K 14/47

(52) U.S. Cl. 514/1.7; 530/324; 514/18.1; 514/15.6; 514/15.4; 514/16.4; 514/17.5; 514/17.6; 514/5.2; 514/10.1; 514/11.3; 514/6.7; 514/11.5; 514/15.7; 514/13.7; 514/19.3; 514/19.4; 514/21.3

(57) ABSTRACT

The present invention relates to novel analogues of neuropeptide Y, pharmaceutical compositions containing the same, pharmaceutical formulations containing the same, and method of treating diseases or conditions mediated by neuropeptide Y-receptor binding. More particularly, the present invention relates to novel analogues of neuropeptide Y having proline substitution at position 34 and other substitution(s) as defined herein that selectively bind to the neuropeptide Y1 receptor subtype compared to the neuropeptide Y2 receptor subtype.

### FIELD OF THE INVENTION

The present invention relates to novel analogues of neuropeptide Y, pharmaceutical compositions containing the same, pharmaceutical formulations containing the same, and method of treating diseases or conditions mediated by neuropeptide Y-receptor binding. More particularly, the present invention relates to novel analogues of neuropeptide Y having proline substitution at position 34 and other substitution(s) as defined herein that selectively bind to the neuropeptide Y1 receptor subtype compared to the neuropeptide Y2 receptor subtype.

### BACKGROUND OF THE INVENTION

Neuropeptide Y (“NPY”), a 36 amino acid peptide neurotransmitter, is a member of the pancreatic family of peptides and shares significant sequence homology with pancreatic polypeptide and peptide YY. Human neuropeptide Y (“hNPY”) has the sequence: H-Tyr-Pro-Ser-Lys-Pro-Asp-Asn-Pro-Gly-Glu-Asp-Ala-Pro-Ala-Glu-Asp-Met-Ala-Arg-Tyr-Tyr-Ser-Ala-Leu-Arg-His-Tyr-Ile-Asn-Leu-Ile-Thr-Arg-Gln-Arg-Tyr-NH₂ (SEQ ID NO:1). NPY was discovered, isolated and sequenced from porcine brain and was named “neuropeptide Y” due to its isolation from neural tissue and the presence of tyrosine as both the amino and carboxy terminal amino acid.

NPY and the other members of its family of peptides all feature a tertiary structure consisting of an N-terminal polyproline helix and an amphiphilic α-helix, connected with a (β-turn, creating a hairpin-like loop, which is sometimes referred to as the “pancreatic polypeptide fold.” The helices are kept together by hydrophobic interactions. The amidated C-terminal end projects away from the hairpin loop.

Subsequent to its discovery, NPY was identified as being the most abundant peptide in the central nervous system with widespread distribution including the cortex, brainstem, hippocampus, hypotahlamus, amygdala, and thalamus, as well as being present in the peripheral nervous system in sympathetic neurons and adrenal chromaffin cells.

NPY seems to fulfill the main neurotransmitter criteria, since it is stored in synaptic granules, is released upon electrical nerve stimulation, and acts at specific receptors. It is clear that NPY is an important messenger in its own right, probably in the brain, where NPY potently inhibits the activity of adenylate cyclase and induces an increase in the intracellular levels of calcium. Central injection of NPY results in blood pressure changes, increased feeding, increased fat storage, elevated blood sugar and insulin, decreased locomotor activity, reduced body temperature, and catalepsy.

NPY appears to interact with a family of closely related receptors. These receptors are generally classified into several subtypes based upon the ability of different tissues and receptors to bind different fragments of neuropeptide Y and the closely related PYY. The Y1 receptor subtype (“NPY-Y1 receptor”) appears to be the major vascular NPY receptor. The Y2 receptor subtype (“NPY-Y2 receptor”) can also occur postjunctionally on vascular smooth muscle. The Y3 receptor subtype (“NPY-Y3 receptor”) appears to be NPY-specific, not binding PYY. This receptor is likely to be present in the adrenal tissues, medulla, heart, and brain stem, among other areas. For a review of neuropeptide Y and neuropeptide Y receptors, see, e.g., C. Wahlestedt and D. Reis, Annual Review of Pharmacology and Toxicology, 33:309-352 (1993). Patent Cooperation Treaty (“PCT”) Publication No. WO 95/00161 describes a series of NPY antagonists and agonists for controlling biological activities such as obesity and cardiovascular function.

European Pat. No. 0759441 and U.S. Pat. No. 5,576,337 report that physiological disorders related to an excess of neuropeptide Y include: disorders or diseases pertaining to the heart, blood vessels or the renal system, such as vasospasm, heart failure, shock, cardiac hypertrophy, increased blood pressure, angina, myocardial infarction, sudden cardiac death, arrythmia, peripheral vascular disease, and abnormal renal conditions such as impaired flow of fluid, abnormal mass transport, or renal failure; conditions related to increased sympathetic nerve activity for example, during or after coronary artery surgery, and operations and surgery in the gastrointestinal tract; cerebral diseases and diseases related to the central nervous system, such as cerebral infarction, neurodegeneration, epilepsy, stroke, and conditions related to stroke, cerebral vasospasm and hemmorrhage, depression, anxiety, schizophrenia, and dementia; conditions related to pain or nociception; diseases related to abnormal gastrointenstinal motility and secretion, such as different forms of ileus, urinary incontinence, and Crohn's disease; abnormal drink and food intake disorders, such as anorexia and metabolic disorders; diseases related to sexual dysfunction and reproductive disorders; conditions or disorders associated with inflammation; respiratory diseases, such as asthma and conditions related to asthma and bronchoconstriction; and diseases related to abnormal hormone release, such as leutinizing hormone, growth hormone, insulin, and prolactin.

PCT Publication No. WO 02/43776 by Reubi reports on the use of compounds that bind the NPY-Y1 receptor for the preparation of a pharmaceutical composition for the diagnosis or treatment of tumors expressing the NPY-Y1 receptor, in particular breast cancer, ovarian cancer and glioblastoma.

There are numerous patents and patent publications that disclose certain NPY analogues and uses thereof, such as U.S. Pat. No. 5,026,685, U.S. Pat. No. 5,328,899, U.S. Pat. No. 6,511,984, PCT Publication No. WO 02/43776, PCT Publication No. WO2007/039318, etc. Notwithstanding the foregoing, there remains a continuing need for NPY analogues having improved potency and/or selectivity and/or in vivo or in vitro characteristics.

### SUMMARY OF THE INVENTION

In one aspect, the present invention provides peptide variants of hNPY of the following formula (I) (SEQ ID NO:2):

(I)
(R²R³)-A¹-A²-A³-A⁴-A⁵-A⁶-A⁷-A⁸-A⁹-A¹⁰-A¹¹-A¹²-A¹³-A¹⁴-A¹⁵-A¹⁶-A¹⁷-A¹⁸-A¹⁹-A²⁰-
A²¹-A²²-A²³-A²⁴-A²⁵-A²⁶-A²⁷-A²⁸-A²⁹-A³⁰-A³¹-A³²-A³³-Pro-A³⁵-A³⁶-A³⁷-R¹

wherein:

A¹ is Tyr, (X¹,X²,X³,X⁴,X⁵)Phe, or HN—CH((CH₂)ₙ-N(R⁴R⁵))—C(O);

A² is Pro, 3Hyp, cis-3Hyp, 4Hyp, or cis-4Hyp;

A³ is Ser, Abu, Aib, Ala, Thr, or HN—CH((CH₂)ₙ—N(R⁴R⁵))—C(O);

A⁴ is Lys, Arg, hArg, Dab, Dap, Orn, or HN—CH((CH₂)ₙ—N(R⁴R⁵))—C(O);

A⁵ is Pro, 3Hyp, cis-3Hyp, 4Hyp, or cis-4Hyp;

A⁶ is Asp, Aib, Asn, Gln, Glu, or HN—CH((CH₂)ₙ—N(R⁴R⁵))—C(O);

A⁷ is Asn, Aib, Gln, or HN—CH((CH₂)ₙ—N(R⁴R⁵))—C(O);

A⁸ is Pro, 3Hyp, cis-3Hyp, 4Hyp, or cis-4Hyp;

A⁹ is Gly, Aib, or HN—CH((CH₂)ₙ—N(R⁴R⁵))—C(O);

A¹⁰ is Glu, Aib, Asn, Asp, Gln, or HN—CH((CH₂)ₙ—N(R⁴R⁵))—C(O);

A¹¹ is Asp, Aib, Asn, Gln, Glu, or HN—CH((CH₂)ₙ—N(R⁴R⁵))—C(O);

A¹² is Ala, Abu, Aib, Nva, Val, or HN—CH((CH₂)ₙ—N(R⁴R⁵))—C(O);

A¹³ is Pro, 3Hyp, cis-3Hyp, 4Hyp, or cis-4Hyp;

A¹⁴ is Ala, Abu, Aib, Nva, Val, or HN—CH((CH₂)ₙ—N(R⁴R⁵))—C(O);

A¹⁵ is Glu, Aib, Asn, Asp, Gln, or HN—CH((CH₂)ₙ—N(R⁴R⁵))—C(O);

A¹⁶ is Asp, Aib, Asn, Gln, Glu, or HN—CH((CH₂)ₙ—N(R⁴R⁵))—C(O);

A¹⁷ is Met, Acc, Aib, Cha, Ile, Leu, hLeu, Nle, Nva, Tle, Val, or HN—CH((CH₂)ₙ—N(R⁴R⁵))—C(O);

A¹⁸ is Ala, Abu, Aib, Nva, Val, or HN—CH((CH₂)ₙ—N(R⁴R⁵))—C(O);

A¹⁹ is Arg, hArg, Apc, Dab, Dap, Lys, Orn, or HN—CH((CH₂)ₙ-N(R⁴R⁵))—C(O);

A²⁰ is Tyr, (X¹,X²,X³,X⁴,X⁵)Phe, or HN—CH((CH₂)ₙ-N(R⁴R⁵))—C(O);

A²¹ is Tyr, (X¹,X²,X³,X⁴,X⁵)Phe, or HN—CH((CH₂)ₙ-N(R⁴R⁵))—C(O);

A²² is Ser, Abu, Aib, Ala, Thr, or HN—CH((CH₂)ₙ—N(R⁴R⁵))—C(O);

A²³ is Ala, Abu, Aib, Nva, Val, or HN—CH((CH₂)ₙ-N(R⁴R⁵))—C(O);

A²⁴ is Leu, Acc, Cha, Ile, hLeu, Nle, Nva, Tle, Val, or HN—CH((CH₂)ₙ—N(R⁴R⁵))—C(O);

A²⁵ is Arg, hArg, Dab, Dap, Lys, Orn, or HN—CH((CH₂)ₙ—N(R⁴R⁵))—C(O);

A²⁶ is H is, 2 Pal, 3 Pal, 4 Pal, or HN—CH((CH₂)ₙ—N(R⁴R⁵))—C(O);

A²⁷ is Tyr, (X¹,X²,X³,X⁴,X⁵)Phe, or HN—CH((CH₂)ₙ-N(R⁴R⁵))—C(O);

A²⁸ is Ile, Acc, Cha, Leu, hLeu, Nle, Nva, Tle, Val, or HN—CH((CH₂)ₙ—N(R⁴R⁵))—C(O);

A²⁹ is Asn, Aib, Gln, or HN—CH((CH₂)ₙ—N(R⁴R⁵))—C(O);

A³⁰ is Leu, Acc, Cha, Ile, hLeu, Nle, Nva, Tle, Val, or HN—CH((CH₂)ₙ—N(R⁴R⁵))—C(O);

A³¹ is Ile, Acc, Cha, Leu, hLeu, Nle, Nva, Tle, Val, or HN—CH((CH₂)ₙ—N(R⁴R⁵))—C(O);

A³² is Thr, Aib, Ser, or HN—CH((CH₂)ₙ—N(R⁴R⁵))—C(O);

A³³ is Arg, hArg, Dab, Dap, Lys, Orn, or HN—CH((CH₂)ₙ—N(R⁴R⁵))—C(O);

A³⁵ is Arg, Aic, Apc, hArg, Dab, Dap, Lys, Orn, NH₂Phe, NH₂CH₂Phe, or HN—CH((CH₂)ₙ—N(R⁴R⁵))—C(O);

A³⁶ is Tyr, Aic, (X¹,X²,X³,X⁴,X⁵)Phe, or HN—CH((CH₂)ₙ-N(R⁴R⁵))—C(O);

A³⁷ is HN—CH((CH₂)ₙ—N(R⁴R⁵))—C(O) or deleted;

R¹ is OH, NH₂, (C₁₋₃₀)alkoxy, or NH—X⁶—CH₂—X⁷, wherein X⁶ is a (C₁₋₄₀)alkyl or (C₂₋₄₀)alkenyl, and wherein X⁷ is H, OH, CO₂H, or C(O)—NH₂;

R² and R³ each is, independently for each occurrence, selected from the group consisting of H, (C₁₋₃₀)alkyl, (C₁₋₃₀)heteroalkyl, (C₁₋₃₀)acyl, (C₂₋₃₀)alkenyl, (C₂₋₃₀)alkynyl, aryl(C₁₋₃₀)alkyl, aryl(C₁₋₃₀)acyl, substituted (C₁₋₃₀)alkyl, substituted (C₁₋₃₀)heteroalkyl, substituted (C₂₋₃₀)acyl, substituted (C₂₋₃₀)alkenyl, substituted (C₂₋₃₀)alkynyl, substituted aryl(C₁₋₃₀)alkyl, and substituted aryl(C₁₋₃₀)acyl;

provided that when R² is (C₁₋₃₀)acyl, aryl(C₁₋₃₀)acyl, substituted (C₂₋₃₀)acyl, or substituted aryl(C₁₋₃₀)acyl, R³ is H, (C₁₋₃₀)alkyl, (C₁₋₃₀)heteroalkyl, (C₂₋₃₀)alkenyl, (C₂₋₃₀)alkynyl, aryl(C₁₋₃₀)alkyl, substituted (C₁₋₃₀)alkyl, substituted (C₁₋₃₀)heteroalkyl, substituted (C₂₋₃₀)alkenyl, substituted (C₂₋₃₀)alkynyl, or substituted aryl(C₁₋₃₀)alkyl;

R⁴ and R⁵ each is, independently for each occurrence, H, (C₁₋₄₀)alkyl, (C₁₋₄₀)heteroalkyl, (C₁₋₄₀)acyl, (C₂₋₄₀)alkenyl, (C₂₋₄₀)alkynyl, aryl(C₁₋₄₀)alkyl, aryl(C₁₋₄₀)acyl, substituted (C₁₋₄₀)alkyl, substituted (C₁₋₄₀)heteroalkyl, substituted (C₁₋₄₀)acyl, substituted (C₂₋₄₀)alkenyl, substituted (C₂₋₄₀)alkynyl, substituted aryl(C₁₋₄₀)alkyl, substituted aryl(C₁₋₄₀)acyl, (C₁₋₄₀)alkylsulfonyl, or C(NH)—NH₂, wherein when R⁴ is (C₁₋₄₀)acyl, aryl(C₁₋₄₀)acyl, substituted (C₁₋₄₀)acyl, substituted aryl(C₁₋₄₀)acyl, (C₁₋₄₀)alkylsulfonyl, or C(NH)—NH₂, then R⁵ is H or (C₁-C₄₀)alkyl, (C₁₋₄₀)heteroalkyl, (C₂₋₄₀)alkenyl, (C₂₋₄₀)alkynyl, aryl(C₁₋₄₀)alkyl, substituted (C₁₋₄₀)alkyl, substituted (C₁₋₄₀)heteroalkyl, substituted (C₂₋₄₀)alkenyl, substituted (C₂₋₄₀)alkynyl, or substituted aryl(C₁₋₄₀)alkyl;

n is, independently for each occurrence, 1, 2, 3, 4, or 5;

X¹, X², X³, X⁴, and X⁵ each is, independently for each occurrence, H, F, Cl, Br, I, (C₁₋₁₀)alkyl, substituted (C₁₋₁₀)alkyl, aryl, substituted aryl, OH, CH₂NH₂, NH₂, NO₂, or CN; and

provided that the compound contains at least one amino acid selected from the group consisting of Aib, Acc, and HN—CH((CH₂)ₙ—N(R⁴R⁵))—C(O) which is not Arg, hArg, Lys, Orn, Dab, or Dap.

A subset (IA) of the compounds covered by the above formula I, are those in which:

A¹ is Tyr or HN—CH((CH₂)ₙ—N(R⁴R⁵))—C(O);

A² is Pro;

A³ is Ser or HN—CH((CH₂)ₙ—N(R⁴R⁵))—C(O);

A⁴ is Lys or HN—CH((CH₂)ₙ—N(R⁴R⁵))—C(O);

A⁵ is Pro;

A⁶ is Asp or HN—CH((CH₂)ₙ—N(R⁴R⁵))—C(O);

A⁷ is Asn or HN—CH((CH₂)ₙ—N(R⁴R⁵))—C(O);

A⁸ is Pro;

A⁹ is Gly or HN—CH((CH₂)ₙ—N(R⁴R⁵))—C(O);

A¹⁰ is Glu or HN—CH((CH₂)ₙ—N(R⁴R⁵))—C(O);

A¹¹ is Asp or HN—CH((CH₂)ₙ—N(R⁴R⁵))—C(O);

A¹² is Ala or HN—CH((CH₂)ₙ—N(R⁴R⁵))—C(O);

A¹³ is Pro;

A¹⁴ is Ala or HN—CH((CH₂)ₙ—N(R⁴R⁵))—C(O);

A¹⁵ is Glu or HN—CH((CH₂)ₙ—N(R⁴R⁵))—C(O);

A¹⁶ is Asp or HN—CH((CH₂)ₙ—N(R⁴R⁵))—C(O);

A¹⁷ is Met or HN—CH((CH₂)ₙ—N(R⁴R⁵))—C(O);

A¹⁸ is Ala or HN—CH((CH₂)ₙ—N(R⁴R⁵))—C(O);

A¹⁹ is Arg or HN—CH((CH₂)ₙ—N(R⁴R⁵))—C(O);

A²⁰ is Tyr or HN—CH((CH₂)ₙ—N(R⁴R⁵))—C(O);

A²¹ Tyr or HN—CH((CH₂)ₙ—N(R⁴R⁵))—C(O);

A²² is Ser or HN—CH((CH₂)ₙ—N(R⁴R⁵))—C(O);

A²³ is Ala or HN—CH((CH₂)ₙ—N(R⁴R⁵))—C(O);

A²⁴ is Leu or HN—CH((CH₂)ₙ—N(R⁴R⁵))—C(O);

A²⁵ is Arg or HN—CH((CH₂)ₙ—N(R⁴R⁵))—C(O);

A²⁶ is H is or HN—CH((CH₂)ₙ—N(R⁴R⁵))—C(O);

A²⁷ is Tyr or HN—CH((CH₂)ₙ—N(R⁴R⁵))—C(O);

A²⁸ is Ile or HN—CH((CH₂)ₙ—N(R⁴R⁵))—C(O);

A²⁹ is Asn or HN—CH((CH₂)ₙ—N(R⁴R⁵))—C(O);

A³⁰ is Leu or HN—CH((CH₂)ₙ—N(R⁴R⁵))—C(O);

A³¹ is Ile, Leu, or HN—CH((CH₂)ₙ—N(R⁴R⁵))—C(O);

A³² is Thr or HN—CH((CH₂)ₙ—N(R⁴R⁵))—C(O);

A³³ is Arg or HN—CH((CH₂)ₙ—N(R⁴R⁵))—C(O);

A³⁵ is Arg or HN—CH₄—CH₂)ₙ—N(R⁴R⁵))—C(O);

A³⁶ is Tyr or HN—CH((CH₂)ₙ—N(R⁴R⁵))—C(O);

A³⁷ is HN—CH((CH₂)ₙ—N(R⁴R⁵))—C(O) or deleted;

R¹ is NH₂;

R² and R³ each is, independently for each occurrence, H or (C₁₋₃₀)acyl;

provided that when R² is (C₁₋₃₀)acyl, R³ is H;

R⁴ and R⁵ each is, independently for each occurrence, H or (C₁₋₄₀)acyl;

n is 4; and

X¹, X², X³, X⁴, and X⁵ each is, independently for each occurrence, H, CH₂NH₂, or

NH₂.

In the subset (IA), HN—CH((CH₂)ₙ—N(R⁴R⁵))—C(O) is preferably Lys(N^{ε}—C(O)—(CH₂)₁₂CH₃).

Preferred compounds of the formula (I) or the subset (IA) are:

Example 1: [Leu³¹, Pro³⁴, Lys³⁶(N^{ε}—C(O)—(CH₂)₁₂—CH₃)]hNPY(1-36)-NH₂; (SEQ ID NO:3)
Example 2: [Leu³¹, Pro³⁴, Lys³⁵(N^{ε}—C(O)—(CH₂)₁₂—CH₃)]hNPY(1-36)-NH₂; (SEQ ID NO:4)
Example 3: [Lys²⁴(N^{ε}—C(O)—(CH₂)₁₂—CH₃), Leu³¹, Pro³⁴]liNPY(1-36)-NH₂; (SEQ ID NO:5)
Example 4: [Lys²³(N^{ε}—C(O)—(CH₂)₁₂—CH₃), Leu³¹, Pro³⁴]hNPY(1-36)-NH₂; (SEQ ID NO:6)
Example 5: [Lys²²(N^{ε}—C(O)—(CH₂)₁₂—CH₃), Leu³¹, Pro³⁴]hNPY(1-36)-NH₂; (SEQ ID NO:7)
Example 6: [Lys²¹(N^{ε}—C(O)—(CH₂)₁₂—CH₃), Leu³¹, Pro³⁴]hNPY(1-36)-NH₂; (SEQ ID NO:8)
Example 7: [Lys²⁰(N^{ε}—C(O)—(CH₂)₁₂—CH₃), Leu³¹, Pro³⁴]hNPY(1-36)-NH₂; (SEQ ID NO:9)
Example 8: [Lys¹⁹(N^{ε}—C(O)—(CH₂)₁₂—CH₃), Leu³¹, Pro³⁴]hNPY(1-36)-NH₂; (SEQ ID NO:10)
Example 9: [Lys¹⁸(N^{ε}—C(O)—(CH₂)₁₂—CH₃), Leu³¹, Pro³⁴]hNPY(1-36)-NH₂; (SEQ ID NO:11)
Example 10: [Lys¹⁷(N^{ε}—C(O)—(CH₂)₁₂—CH₃), Leu³¹, Pro³⁴]hNPY(1-36)-NH₂; (SEQ ID NO:12)
Example 11: [Lys¹⁶(N^{ε}—C(O)—(CH₂)₁₂—CH₃), Leu³¹, Pro³⁴]hNPY(1-36)-NH₂; (SEQ ID NO:13)
Example 12: [Lys¹⁵(N^{ε}—C(O)—(CH₂)₁₂—CH₃), Leu³¹, Pro³⁴]hNPY(1-36)-NH₂; (SEQ ID NO:14)
Example 13: [Lys¹⁴(N^{ε}—C(O)—(CH₂)₁₂—CH₃), Leu³¹, Pro³⁴]hNPY(1-36)-NH₂; (SEQ ID NO:15)
Example 14: [Lys¹²(N^{ε}—C(O)—(CH₂)₁₂—CH₃), Leu³¹, Pro³⁴]hNPY(1-36)-NH₂; (SEQ ID NO:16)
Example 15: [Lys¹¹(N^{ε}—C(O)—(CH₂)₁₂—CH₃), Leu³¹, Pro³⁴]hNPY(1-36)-NH₂; (SEQ ID NO:17)
Example 16: [Lys¹⁰(N^{ε}—C(O)—(CH₂)₁₂—CH₃), Leu³¹, Pro³⁴]hNPY(1-36)-NH₂; (SEQ ID NO:18)
Example 17: [Lys⁹(N^{ε}—C(O)—(CH₂)₁₂—CH₃), Leu³¹, Pro³⁴]hNPY(1-36)-NH₂; (SEQ ID NO:19)
Example 18: [Lys⁷(N^{ε}—C(O)—(CH₂)₁₂—CH₃), Leu³¹, Pro³⁴]hNPY(1-36)-NH₂; (SEQ ID NO:20)
Example 19: [Lys⁶(N^{ε}—C(O)—(CH₂)₁₂—CH₃), Leu³¹, Pro³⁴]hNPY(1-36)-NH₂; (SEQ ID NO:21)
Example 20: [Lys⁴(N^{ε}—C(O)—(CH₂)₁₂—CH₃), Leu³¹, Pro³⁴]hNPY(1-36)-NH₂; (SEQ ID NO:22)
Example 21: [Lys³(N^{ε}—C(O)—(CH₂)₁₂—CH₃), Leu³¹, Pro³⁴]hNPY(1-36)-NH₂; (SEQ ID NO:23)
Example 22: [Lys^{l}(N^{ε}—C(O)—(CH₂)₁₂—CH₃), Leu³¹, Pro³⁴]hNPY(1-36)-NH₂; (SEQ ID NO:24)
Example 23: [Leu³¹, Pro³⁴, Lys³⁷(N^{ε}—C(O)—(CH₂)₁₂—CH₃)]hNPY(1-37)-NH₂; (SEQ ID NO:25)
Example 24: [Leu³¹, Lys³³(N^{ε}—C(O)—(CH₂)₁₂—CH₃), Pro³⁴]hNPY(1-36)-NH₂; (SEQ ID NO:26)
Example 25: [Leu³¹, Lys³²(N^{ε}—C(O)—(CH₂)₁₂—CH₃), Pro³⁴]hNPY(1-36)-NH₂; (SEQ ID NO:27)
Example 26: [Lys³¹(N^{ε}—C(O)—(CH₂)₁₂—CH₃), Pro³⁴]hNPY(1-36)-NH₂; (SEQ ID NO:28)
Example 27: [Lys³⁰(N^{ε}—C(O)—(CH₂)₁₂—CH₃), Leu³¹, Pro³⁴]hNPY(1-36)-NH₂; (SEQ ID NO:29)
Example 28: [Lys²⁹(N^{ε}—C(O)—(CH₂)₁₂—CH₃), Leu³¹, Pro³⁴]hNPY(1-36)-NH₂; (SEQ ID NO:30)
Example 29: [Lys²⁸(N^{ε}—C(O)—(CH₂)₁₂—CH₃), Leu³¹, Pro³⁴]hNPY(1-36)-NH₂; (SEQ ID NO:31)
Example 30: [Lys²⁷(N^{ε}—C(O)—(CH₂)₁₂—CH₃), Leu³¹, Pro³⁴]hNPY(1-36)-NH₂; (SEQ ID NO:32)
Example 31: [Lys²⁶(N^{ε}—C(O)—(CH₂)₁₂—CH₃), Leu³¹, Pro³⁴]hNPY(1-36)-NH₂; (SEQ ID NO:33)
Example 32: [Lys²⁵(N^{ε}—C(O)—(CH₂)₁₂—CH₃), Leu³¹, Pro³⁴]hNPY(1-36)-NH₂; (SEQ ID NO:34) and
Example 33: [CH₃(CH₂)₈(CO)-Tyr¹, Nle¹⁷, Pro³⁴]hNPY(1-36)-NH₂. (SEQ ID NO:37)

### DETAILED DESCRIPTION OF THE INVENTION

As used herein the term “amino acid” refers to any natural or unnatural amino acid, including but not limited to α-amino acids, β-amino acids, or γ-amino acids, and may be either D- or L-amino acid unless otherwise indicated.

With the exception of the N-terminal amino acid, all amino acid abbreviations (e.g., Ala) in this disclosure have the structure —NH—C(R)(R′)—CO—, wherein R and R′ each is, independently, hydrogen or the side chain of an amino acid (e.g., R═CH₃ and R′═H for Ala), or R and R′ may be joined to form a ring system. For the N-terminal amino acid, the abbreviation stands for the structure of (R²R³)—N—C(R)(R′)—CO—, wherein R² and R³ are as defined in the formula (I).

A peptide of this invention is also denoted by another format, e.g., [Pro³⁴]hNPY(1-36)—NH₂ (SEQ ID NO:1), with the substituted amino acids from the natural sequence placed between the brackets, e.g., Pro for Gln in hNPY. The designation “NH₂” in hNPY(1-36)-NH₂ (SEQ ID NO:1) indicates that the C-terminus of the peptide is amidated whereas hNPY(1-36)-OH (SEQ ID NO:36) indicates the free acid form.

The following list of some of the abbreviations used in the present application is provided for ease of reference, however, any abbreviation used in the instant application not defined herein are not used contrary to the recognized meanings thereof.
Abu α-aminobutyric acid
Acc 1-amino-1-cyclo(C₃₋₉)alkyl carboxylic acid, wherein
   A3c represents 1-amino-1-cyclopropanecarboxylic acid;
   A4c represents 1-amino-1-cyclobutanecarboxylic acid;
   A5 c represents 1-amino-1-cyclopentanecarboxylic acid; and
   A6c represents 1-amino-1-cyclohexanecarboxylic acid
Adc 10-aminodecanoic acid
Ado 12-aminododecanoic acid
Ahp 7-aminoheptanoic acid
Ahx 6-aminohexanoic acid
Aib α-aminoisobutyric acid
Aic 2-aminoindan-2-carboxylic acid
Ala or A alanine
Anc 9-aminononanoic acid
Aoc 8-aminooctanoic acid
Apc 4-amino-4-carboxypiperidine, represented by structure:

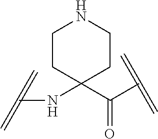

wherein, the parallel lines “ [symbol] ” indicate points of attachment of the moiety to another moiety or sequence.
Apn 5-aminopentanoic acid
Arg or R arginine
hArg homoarginine
Asn or N asparagine
Asp or D aspartic acid
Aun 11-aminoundecanoic acid
Cha β-cyclohexylalanine
Cys or C cysteine
Dab 2,4-diaminobutyric acid
Dap 2,3-diaminopropionic acid
Dhp 3,4-dehydroproline
Dmt 5,5-dimethylthiazolidine-4-carboxylic acid
Gaba 4-aminobutyric acid
Gln or Q glutamine
Glu or E glutamic acid
Gly or G glycine
H is or H histidine
3Hyp trans-3-hydroxy-L-proline, i.e., (2S,3S)-3-hydroxypyrrolidine-2-carboxylic acid
cis-3Hyp cis-3-hydroxy-L-proline, i.e., (2S,3R)-3-hydroxypyrrolidine-2-carboxylic acid
4Hyp 4-hydroxyproline, i.e., (2S,4R)-4-hydroxypyrrolidine-2-carboxylic acid
cis-4Hyp cis-4-hydroxy-L-proline, i.e., (2S,4S)-4-hydroxypyrrolidine-2-carboxylic acid
Ile or I isoleucine
Inc indoline-2-carboxylic acid
Inp isonipecotic acid
Ktp 4-ketoproline
Leu or L leucine
hLeu homoleucine
Lys or K lysine
Met or M methionine
Nip nipecotic acid

Nle norleucine
N^{ε} indicates that the entity within the parentheses is coupled to the epsilon-nitrogen of the Lys sidechain
Nva norvaline
Oic octahydroindole-2-carboxylic acid
Orn ornithine
2-Pal β-(2-pyridyl)alanine
3-Pal β-(3-pyridyl)alanine
4-Pal β-(4-pyridyl)alanine
Phe or F phenylalanine
hPhe homophenylalanine
4NH₂CH₂Phe 4-aminomethyl-phenylalanine
4NH₂Phe 4-amino-phenylalanine
Pro or P proline
hPro homoproline
Sar sarcosine or N-methyl glycine
Ser or S serine
Thr or T threonine
Tic 1,2,3,4-tetrahydroisoquinoline-3-carboxylic acid
Tle tert-leucine
Val or V valine

“Alkyl” refers to a hydrocarbon group containing one or more carbon atoms, where multiple carbon atoms if present are joined by single bonds, examples of which include but are not limited to methyl, ethyl, propyl and butyl. The alkyl hydrocarbon group may be straight-chain or contain one or more branches or cyclic groups, examples of which include, but are not limited to, isopropyl and tertbutyl.

“Substituted alkyl” refers to an alkyl wherein one or more hydrogen atoms of the hydrocarbon group are replaced with one or more substituents selected from the group consisting of halogen, (i.e., fluorine, chlorine, bromine, and iodine), OH, CN, SH, NH₂, NHCH₃, NO₂, (C₁₋₂)alkyl substituted with 1 to 6 halogens, CF₃, OCH₃, OCF₃, and (CH₂)₀₋₄—COOH. In different embodiments, 1, 2, 3 or 4 substituents are present. The presence of (CH₂)₀₋₄—COOH results in the production of an alkyl acid. Examples of alkyl acids containing (CH₂)₀₋₄—COOH include, but are not limited to, 2-norbornane acetic acid, tert-butyric acid and 3-cyclopentyl propionic acid.

“Heteroalkyl” refers to an alkyl wherein one of more of the carbon atoms in the hydrocarbon group are replaced with one or more of the following atoms or groups: amino, amido, O, S, N, and carbonyl. In different embodiments, 1 or 2 heteroatoms are present.

“Substituted heteroalkyl” refers to a heteroalkyl wherein one or more hydrogen atoms of the hydrocarbon group are replaced with one or more substituents selected from the group consisting of halogen, (i.e., fluorine, chlorine, bromine, and iodine), OH, CN, SH, NH₂, NHCH₃, NO₂, (C₁₋₂)alkyl substituted with 1 to 6 halogens, CF₃, OCH₃, OCF₃, and (CH₂)₀₋₄—COOH. In different embodiments, 1, 2, 3 or 4 substituents are present.

“Alkenyl” refers to a hydrocarbon group made up of two or more carbons where one or more carbon-carbon double bonds are present, examples of which include, but are not limited to, vinyl, allyl, butenyl and propenyl. The alkenyl hydrocarbon group may be straight-chain or contain one or more branches or cyclic groups, examples of which include, but are not limited to, n-butenyl versus t-butenyl, and n-pentenyl compared to cyclopentenyl.

“Substituted alkenyl” refers to an alkenyl wherein one or more hydrogens are replaced with one or more substituents selected from the group consisting of halogen (i.e., fluorine, chlorine, bromine, and iodine), OH, CN, SH, NH₂, NHCH₃, NO₂, (C₁₋₂)alkyl substituted with 1 to 6 halogens, CF₃, OCH₃, OCF₃, and (CH₂)₀₋₄—COOH. In different embodiments, 1, 2, 3 or 4 substituents are present.

“Aryl” refers to an optionally substituted aromatic group with at least one ring having a conjugated π-electron system containing up to two conjugated or fused ring systems. Aryl includes, but is not limited to, carboxylic aryl, heterocyclic aryl and biaryl groups. Preferably, an aryl is a 5- or 6-membered ring. Preferred atoms for a heterocyclic aryl include, but are not limited to, one or more of sulfur, oxygen and nitrogen. Examples of aryl include, but are not limited to, phenyl, 1-naphthyl, 2-naphthyl, indole, quinoline, 2-imidazole, and 9-anthracene. Aryl substituents are selected from the group consisting of (C₁₋₄ alkyl, (C₁₋₄ alkoxy, halogen (i.e., fluorine, chlorine, bromine, and iodine), OH, CN, SH, NH₂, NO₂, (C₁₋₂)alkyl substituted with 1 to 5 halogens, CF₃, OCF₃, and (CH₂)₀₋₄—COOH. In different embodiments, aryl contains 0, 1, 2, 3 or 4 substituents.

“Alkylaryl” refers to an “alkyl” joined to an “aryl,” as defined above.

The term “cycloalkyl” is intended to include a mono-cycloalkyl group or a bi-cycloalkyl group of the indicated carbon number known to those of skill in the art.

The term “heterocycle” includes mono-cyclic and bi-cyclic systems having one or more heteroatoms, such as oxygen, nitrogen and sulfur. The ring systems may be aromatic, for example, pyridine, indole, quinoline, pyrimidine, thiophene (also known as thienyl), furan, benzothiophene, tetrazole, dihydroindole, indazole, N-formylindole, benzimidazole, thiazole, and thiadiazole. The ring systems also may be non-aromatic, for example, but not limited to, pyrrolidine, piperidine, morpholine, and the like.

### Synthesis

The compounds of this invention can be and were produced using the techniques disclosed in the examples herein as well as techniques that are well known in the art. For example, a polypeptide region of an NPY analogue can be chemically or biochemically synthesized and/or modified. See, e.g., Stewart, J. M., et al., Solid Phase Synthesis, Pierce Chemical Co., 2d ed. (1984); and see, e.g., Sambrook et al., Molecular Cloning, A Laboratory Manual, 2^{nd} ed., Cold Spring Harbor Laboratory Press (1989) for examples of techniques for biochemical synthesis involving the introduction of a nucleic acid into a cell and expression of nucleic acids.

Physical data for the compounds exemplified herein are given in Table 1.

TABLE 1

| Example | Mol. Wt. | Mol. Wt. | % Purity |
|---|---|---|---|
| Number | (Expected) | (ESI-MS) | (HPLC) |
| 1 | 4416.1 | 4415.9 | >99 |
| 2 | 4423.1 | 4423.4 | >99 |
| 3 | 4466.1 | 4466.1 | >98 |
| 4 | 4508.2 | 4508.2 | >98 |
| 5 | 4492.2 | 4491.9 | >99 |
| 6 | 4416.1 | 4416.4 | >99 |
| 7 | 4416.1 | 4416.5 | >99 |
| 8 | 4423.1 | 4423.2 | >97 |
| 9 | 4508.2 | 4508.4 | >99 |
| 10 | 4448.0 | 4448.2 | >99 |
| 11 | 4464.2 | 4463.7 | >99 |
| 12 | 4450.1 | 4450.5 | >99 |
| 13 | 4508.2 | 4508.3 | >99 |
| 14 | 4508.2 | 4508.4 | >99 |
| 15 | 4464.2 | 4464.9 | >99 |
| 16 | 4450.1 | 4450.3 | >99 |
| 17 | 4522.2 | 4522.2 | >99 |
| 18 | 4465.1 | 4465.1 | >99 |
| 19 | 4464.2 | 4464.2 | >99 |
| 20 | 4451.1 | 4451.2 | >99 |
| 21 | 4492.2 | 4492.0 | >99 |
| 22 | 4416.1 | 4416.3 | >99 |
| 23 | 4579.2 | 4579.2 | >99 |
| 24 | 4423.1 | 4422.8 | >99 |
| 25 | 4478.1 | 4478.1 | >99 |
| 26 | 4466.1 | 4466.3 | >99 |
| 27 | 4466.1 | 4466.3 | >99 |
| 28 | 4465.1 | 4465.2 | >99 |
| 29 | 4466.1 | 4466.1 | >99 |
| 30 | 4416.1 | 4416.4 | >99 |
| 31 | 4442.1 | 4442.4 | >99 |
| 32 | 4423.1 | 4423.2 | >99 |
| 33 | 4376.9 | 4377.4 | 95.2 |

### In Vitro Radioligand NPY-Y1 and NPY-Y2 Receptor Binding Assays

Human neuroblastoma cell lines, SK-N-MC and SK-N-BE2 (American Type Culture Collection, Rockville, Md., USA), expressing the NPY-Y1 and NPY-Y2 receptors, respectifully, were cultured in EMEM containing 10% fetal calf serum and 5% chicken embryo extract, and maintained at 37° C. in a humidifed atmosphere of and 95% air and 5% CO₂.

For the in vitro NPY-Y1 and NPY-Y2 radioligand binding assays, the appropriate cells (SK-N-MC for NPY-Y1; SK-N-BE2 for NPY-Y2) were harvested, homogenized in 20 ml of ice-cold 50 mM Tris-HCl with a Brinkman Polytron (Westbury, N.Y., USA) (setting 6, sec). The homogenates were washed twice by centrifugation (39,000 g/10 min), and the final pellets were resuspended in 50 mM Tris-HCl, containing 2.5 mM MgCl₂, 0.1 mg/ml bacitracin (Sigma Chemical, St. Louis, Mo., USA), and 0.1% BSA.

For assay, aliquots (0.4 ml) of the foregoing suspensions were incubated with 0.05 nM [¹²⁵I]PYY (2200 Ci/mmol, Perkin-Elmer, Boston, Mass.), with and without 0.05 ml of unlabeled competing test peptides. After a 100 min incubation (25° C.), the bound [¹²⁵I]PYY was separated from the free by rapid filtration through GF/C filters (Brandel, Gaithersburg, Md., USA), which had been previously soaked in 0.3% polyethyleneimine. The filters were then washed three times with 5-ml aliquots of ice-cold 50 mM Tris-HCl, and the bound radioactivity trapped on the filters was counted by gamma spectrometry (Wallac LKB, Gaithersburg, Md., USA). Specific binding was defined as the total [¹²⁵I]PYY bound minus that bound in the presence of 1000 nM PYY (Bachem, Torrence, Calif., USA). Inhibition constants (Ki) were calculated using the well-known Cheng-Prusoff equation, and said data, together with selectivity of said compounds with respect to the NPY-Y1 and the NPY-Y2, are given in Table 2.

Each of the compounds of Examples 1-32 was subjected to the immediately foregoing radioligand assays, and nearly all of said compounds were found to have Ki of under 100 nM, as well as some of the exemplified compounds having Ki values in sub-nM range. It was also found that nearly all of said compounds highly selectively bind to the NPY-Y1 compared to the NPY-Y2.

TABLE 2

| Example No. | Ki (nM) for Y1 | Ki (nM) for Y2 | Selectivity |
|---|---|---|---|
| 1 | 307.67 | 367 | Y1 |
| 2 | 120.44 | 643 | Y1 |
| 3 | 3.56 | 668 | Y1 |
| 4 | 19.67 | >1000 | Y1 |
| 5 | 4.79 | 133 | Y1 |
| 6 | 10.65 | 19 | Y1 |
| 7 | 108.38 | 13 | Y2 |
| 8 | 13.66 | 15 | Y1 |
| 9 | 6.68 | 10 | Y1 |
| 10 | 54.26 | 11 | Y2 |
| 11 | 20.35 | 26 | Y1 |
| 12 | 10.00 | 611 | Y1 |
| 13 | 6.18 | 383 | Y1 |
| 14 | 22.50 | 270 | Y1 |
| 15 | 4.79 | 40 | Y1 |
| 16 | 5.68 | 23 | Y1 |
| 17 | 11.82 | 41 | Y1 |
| 18 | 2.94 | 74 | Y1 |
| 19 | 3.44 | 33 | Y1 |
| 20 | 1.49 | 75 | Y1 |
| 21 | 0.55 | 138 | Y1 |
| 22 | 0.73 | 80 | Y1 |
| 23 | N/A | N/A | N/A |
| 24 | N/A | N/A | N/A |
| 25 | 87.47 | 227 | Y1 |
| 26 | 35.17 | >1000 | Y1 |
| 27 | 10.35 | 292 | Y1 |
| 28 | 29.67 | 267 | Y1 |
| 29 | 53.58 | >1000 | Y1 |
| 30 | 187.50 | 787 | Y1 |
| 31 | 8.24 | 107 | Y1 |
| 32 | 21.57 | >1000 | Y1 |
| 33 | 0.84 | 672 | Y1 |

### Administration

The peptides of this invention can be provided in the form of pharmaceutically acceptable salts. Examples of such salts include, but are not limited to, those formed with organic acids (e.g., acetic, lactic, maleic, citric, malic, ascorbic, succinic, benzoic, methanesulfonic, toluenesulfonic, or pamoic acid), inorganic acids (e.g., hydrochloric acid, sulfuric acid, or phosphoric acid), and polymeric acids (e.g., tannic acid, carboxymethyl cellulose, polylactic, polyglycolic, or copolymers of polylactic-glycolic acids). A typical method of making a salt of a peptide of the present invention is well known in the art and can be accomplished by standard methods of salt exchange. Accordingly, the TFA salt of a peptide of the present invention (the TFA salt results from the purification of the peptide by using preparative HPLC eluting with TFA containing buffer solutions) can be converted into another salt, such as an acetate salt, by dissolving the peptide in a small amount of 0.25 N acetic acid aqueous solution. The resulting solution is applied to a semi-prep HPLC column (Zorbax, 300 SB, C-8). The column is eluted with (1) 0.1N ammonium acetate aqueous solution for 0.5 hours, (2) 0.25N acetic acid aqueous solution for 0.5 hours, and (3) a linear gradient (20% to 100% of solution B over 30 min) at a flow rate of 4 ml/min (solution A is 0.25N acetic acid aqueous solution; solution B is 0.25N acetic acid in acetonitrile/water, 80:20). The fractions containing the peptide are collected and lyophilized to dryness.

The dosage of active ingredient in the compositions of this invention may be varied, however, it is necessary that the amount of the active ingredient be such that a suitable dosage form is obtained. The selected dosage depends upon the desired therapeutic effect, the route of administration, and the duration of the treatment. In general, an effective dosage for the activities of this invention is in the range of 1×10⁻⁷ to 200 mg/kg/day, preferably 1×10⁻⁴ to 100 mg/kg/day, which can be administered as a single dose or divided into multiple doses.

The compounds of this invention can be administered by oral, parenteral (e.g., intramuscular, intraperitoneal, intravenous or subcutaneous injection, or implant), nasal, vaginal, rectal, sublingual, or topical routes of administration, and can be formulated with pharmaceutically acceptable carriers to provide dosage forms appropriate for each route of administration.

Solid dosage forms for oral administration include capsules, tablets, pills, powders and granules. In such solid dosage forms, the active compound is admixed with at least one inert pharmaceutically acceptable carrier such as sucrose, lactose, or starch. Such dosage forms can also comprise, as is normal practice, additional substances other than such inert diluents, e.g., lubricating agents such as magnesium stearate. In the case of capsules, tablets and pills, the dosage forms may also comprise buffering agents. Tablets and pills can additionally be prepared with enteric coatings.

Liquid dosage forms for oral administration include, without limitation, pharmaceutically acceptable emulsions, solutions, suspensions, syrups, elixirs, and the like, containing inert diluents commonly used in the art, such as water. Besides such inert diluents, compositions can also include adjuvants, such as wetting agents, emulsifying and suspending agents, and sweetening, flavoring and perfuming agents.

Preparations according to this invention for parenteral administration include, without limitation, sterile aqueous or non-aqueous solutions, suspensions, emulsions, and the like. Examples of non-aqueous solvents or vehicles include propylene glycol, polyethylene glycol, vegetable oils, such as olive oil and corn oil, gelatin, and injectable organic esters such as ethyl oleate. Such dosage forms may also contain adjuvants such as preserving, wetting, emulsifying, and dispersing agents. They may be sterilized by, for example, filtering through a bacteria-retaining filter, incorporating sterilizing agents, irradiating, or heating the compositions. They can also be manufactured in the form of sterile solid compositions which can be dissolved in sterile water, or some other sterile injectable medium, immediately before use.

Compositions for rectal or vaginal administration are preferably suppositories which may contain, in addition to the active substance, excipients such as coca butter or a suppository wax.

Compositions for nasal or sublingual administration are also prepared with standard excipients well known in the art.

Further, a compound of this invention can be administered in a sustained release composition such as those described in the following patents and patent applications. U.S. Pat. No. 5,672,659 teaches sustained release compositions comprising a bioactive agent and a polyester. U.S. Pat. No. 5,595,760 teaches sustained release compositions comprising a bioactive agent in a gelable form. U.S. Pat. No. 5,821,221 teaches polymeric sustained release compositions comprising a bioactive agent and chitosan. U.S. Pat. No. 5,916,883 teaches sustained release compositions comprising a bioactive agent and cyclodextrin. PCT publication WO99/38536 teaches absorbable sustained release compositions of a bioactive agent. PCT publication WO00/04916 teaches a process for making microparticles comprising a therapeutic agent such as a peptide in an oil-in-water process. PCT publication WO00/09166 teaches complexes comprising a therapeutic agent such as a peptide and a phosphorylated polymer. PCT publication WO00/25826 teaches complexes comprising a therapeutic agent such as a peptide and a polymer bearing a non-polymerizable lactone.

Unless defined otherwise, all technical and scientific terms used herein have the same meaning as commonly understood by one of ordinary skill in the art to which this invention belongs. Also, all publications, patent applications, patents and other references mentioned herein are hereby incorporated by reference, each in its entirety.

What is claimed is:

1. A compound according to formula (I) (SEQ ID NO:2):
(I)
(R²R³)-A¹-A²-A³-A⁴-A⁵-A⁶-A⁷-A⁸-A⁹-A¹⁰-A¹¹-A¹²-A¹³-A¹⁴-A¹⁵-A¹⁶-A¹⁷-A¹⁸-A¹⁹-A²⁰-A²¹-
A²²-A²³-A²⁴-A²⁵-A²⁶-A²⁷-A²⁸-A²⁹-A³⁰-A³¹-A³²-A³³-Pro-A³⁵-A³⁶-A³⁷-R¹
wherein:
A¹ is Tyr, (X¹,X²,X³,X⁴,X⁵)Phe, or HN—CH((CH₂)ₙ—N(R⁴R⁵))—C(O);
A² is Pro, 3Hyp, cis-3Hyp, 4Hyp, or cis-4Hyp;
A³ is Ser, Abu, Aib, Ala, Thr, or HN—CH((CH₂)ₙ—N(R⁴R⁵))—C(O);
A⁴ is Lys, Arg, hArg, Dab, Dap, Orn, or HN—CH((CH₂)ₙ—N(R⁴R⁵))—C(O);
A⁵ is Pro, 3Hyp, cis-3Hyp, 4Hyp, or cis-4Hyp;
A⁶ is Asp, Aib, Asn, Gln, Glu, or HN—CH((CH₂)ₙ—N(R⁴R⁵))—C(O);
A⁷ is Asn, Aib, Gln, or HN—CH((CH₂)ₙ—N(R⁴R⁵))—C(O);
A⁸ is Pro, 3Hyp, cis-3Hyp, 4Hyp, or cis-4Hyp;
A⁹ is Gly, Aib, or HN—CH((CH₂)ₙ—N(R⁴R⁵))—C(O);
A¹⁰ is Glu, Aib, Asn, Asp, Gln, or HN—CH((CH₂)ₙ—N(R⁴R⁵))—C(O);
A¹¹ is Asp, Aib, Asn, Gln, Glu, or HN—CH((CH₂)ₙ—N(R⁴R⁵))—C(O);
A¹² is Ala, Abu, Aib, Nva, Val, or HN—CH((CH₂)ₙ—N(R⁴R⁵))—C(O);
A¹³ is Pro, 3Hyp, cis-3Hyp, 4Hyp, or cis-4Hyp;
A¹⁴ is Ala, Abu, Aib, Nva, Val, or HN—CH((CH₂)ₙ—N(R⁴R⁵))—C(O);
A¹⁵ is Glu, Aib, Asn, Asp, Gln, or HN—CH((CH₂)ₙ—N(R⁴R⁵))—C(O);
A¹⁶ is Asp, Aib, Asn, Gln, Glu, or HN—CH((CH₂)ₙ—N(R⁴R⁵))—C(O);
A¹⁷ is Met, Acc, Aib, Cha, Ile, Leu, hLeu, Nle, Nva, Tle, Val, or HN—CH((CH₂)ₙ—N(R⁴R⁵))—C(O);
A¹⁸ is Ala, Abu, Aib, Nva, Val, or HN—CH((CH₂)ₙ—N(R⁴R⁵))—C(O);
A¹⁹ is Arg, hArg, Apc, Dab, Dap, Lys, Orn, or HN—CH((CH₂)ₙ—N(R⁴R⁵))—C(O);
A²⁰ is Tyr, (X¹,X²,X³,X⁴,X⁵)Phe, or HN—CH((CH₂)ₙ—N(R⁴R⁵))—C(O);
A²¹ is Tyr, (X¹,X²,X³,X⁴,X⁵)Phe, or HN—CH((CH₂)ₙ—N(R⁴R⁵))—C(O);
A²² is Ser, Abu, Aib, Ala, Thr, or HN—CH((CH₂)ₙ—N(R⁴R⁵))—C(O);
A²³ is Ala, Abu, Aib, Nva, Val, or HN—CH((CH₂)ₙ—N(R⁴R⁵))—C(O);
A²⁴ is Leu, Acc, Cha, Ile, hLeu, Nle, Nva, Tle, Val, or HN—CH((CH₂)ₙ—N(R⁴R⁵))—C(O);
A²⁵ is Arg, hArg, Dab, Dap, Lys, Orn, or HN—CH((CH₂)ₙ—N(R⁴R⁵))—C(O);
A²⁶ is H is, 2 Pal, 3 Pal, 4 Pal, or HN—CH((CH₂)ₙ—N(R⁴R⁵))—C(O);
A²⁷ is Tyr, (X¹,X²,X³,X⁴,X⁵)Phe, or HN—CH((CH₂)ₙ—N(R⁴R⁵))—C(O);
A²⁸ is Ile, Acc, Cha, Leu, hLeu, Nle, Nva, Tle, Val, or HN—CH((CH₂)ₙ—N(R⁴R⁵))—C(O);
A²⁹ is Asn, Aib, Gln, or HN—CH((CH₂)ₙ—N(R⁴R⁵))—C(O);
A³⁰ is Leu, Acc, Cha, Ile, hLeu, Nle, Nva, Tle, Val, or HN—CH((CH₂)ₙ—N(R⁴R⁵))—C(O);
A³¹ is Ile, Acc, Cha, Leu, hLeu, Nle, Nva, Tle, Val, or HN—CH((CH₂)ₙ—N(R⁴R⁵))—C(O);
A³² is Thr, Aib, Ser, or HN—CH((CH₂)ₙ—N(R⁴R⁵))—C(O);
A³³ is Arg, hArg, Dab, Dap, Lys, Orn, or HN—CH((CH₂)ₙ—N(R⁴R⁵))—C(O);
A³⁵ is Arg, Aic, Apc, hArg, Dab, Dap, Lys, Orn, NH₂Phe, NH₂CH₂Phe, or HN—CH((CH₂)ₙ—N(R⁴R⁵))—C(O);
A³⁶ is Tyr, Aic, (X¹,X²,X³,X⁴,X⁵)Phe, or HN—CH((CH₂)ₙ—N(R⁴R⁵))—C(O);
A³⁷ is HN—CH((CH₂)ₙ—N(R⁴R⁵))—C(O) or deleted;
R¹ is OH, NH₂, (C₁₋₃₀)alkoxy, or NH—X⁶—CH₂—X⁷, wherein X⁶ is a (C₁₋₄₀)alkyl or (C₂₋₄₀)alkenyl, and wherein X⁷ is H, OH, CO₂H, or C(O)—NH₂;
R² and R³ each is, independently for each occurrence, selected from the group consisting of H, (C₁₋₃₀)alkyl, (C₁₋₃₀)heteroalkyl, (C₁₋₃₀)acyl, (C₂₋₃₀)alkenyl, (C₂₋₃₀)alkynyl, aryl(C₁₋₃₀)alkyl, aryl(C₁₋₃₀)acyl, substituted (C₁₋₃₀)alkyl, substituted (C₁₋₃₀)heteroalkyl, substituted (C₂₋₃₀)acyl, substituted (C₂₋₃₀)alkenyl, substituted (C₂₋₃₀)alkynyl, substituted aryl(C₁₋₃₀)alkyl, and substituted aryl(C₁₋₃₀)acyl;
provided that when R² is (C₁₋₃₀)acyl, aryl(C₁₋₃₀)acyl, substituted (C₂₋₃₀)acyl, or substituted aryl(C₁₋₃₀)acyl, R³ is H, (C₁₋₃₀)alkyl, (C₁₋₃₀)heteroalkyl, (C₂₋₃₀)alkenyl, (C₂₋₃₀)alkynyl, aryl(C₁₋₃₀)alkyl, substituted (C₁₋₃₀)alkyl, substituted (C₁₋₃₀)heteroalkyl, substituted (C₂₋₃₀)alkenyl, substituted (C₂₋₃₀)alkynyl, or substituted aryl(C₁₋₃₀)alkyl;
R⁴ and R⁵ each is, independently for each occurrence, H, (C₁₋₄₀)alkyl, (C₁₋₄₀)heteroalkyl, (C₁₋₄₀)acyl, (C₂₋₄₀)alkenyl, (C₂₋₄₀)alkynyl, aryl(C₁₋₄₀)alkyl, aryl(C₁₋₄₀)acyl, substituted (C₁₋₄₀)alkyl, substituted (C₁₋₄₀)heteroalkyl, substituted (C₁₋₄₀)acyl, substituted (C₂₋₄₀)alkenyl, substituted (C₂₋₄₀)alkynyl, substituted aryl(C₁₋₄₀)alkyl, substituted aryl(C₁₋₄₀)acyl, (C₁₋₄₀)alkylsulfonyl, or C(NH)—NH₂, wherein when R⁴ is (C₁₋₄₀)acyl, aryl(C₁₋₄₀)acyl, substituted (C₁₋₄₀)acyl, substituted aryl(C₁₋₄₀)acyl, (0-40)alkylsulfonyl, or C(NH)—NH₂, then R⁵ is H or (C₁-C₄₀)alkyl, (C₁₋₄₀)heteroalkyl, (C₂₋₄₀)alkenyl, (C₂₋₄₀)alkynyl, aryl(C₁₋₄₀)alkyl, substituted (C₁₋₄₀)alkyl, substituted (C₁₋₄₀)heteroalkyl, substituted (C₂₋₄₀)alkenyl, substituted (C₂₋₄₀)alkynyl, or substituted aryl(C₁₋₄₀)alkyl;
n is, independently for each occurrence, 1, 2, 3, 4, or 5;
X¹, X², X³, X⁴, and X⁵ each is, independently for each occurrence, H, F, Cl, Br, I, (0-10)alkyl, substituted (C₁₋₁₀)alkyl, aryl, substituted aryl, OH, CH₂NH₂, NH₂, NO₂, or CN; and
provided that the compound contains at least one amino acid selected from the group consisting of Aib, Acc, and HN—CH((CH₂)ₙ—N(R⁴R⁵))—C(O) which is not Arg, hArg, Lys, Orn, Dab, or Dap;
or a pharmaceutically acceptable salt thereof.

2. A compound according to claim 1, wherein:
A¹ is Tyr or HN—CH((CH₂)ₙ—N(R⁴R⁵))—C(O);
A² is Pro;
A³ is Ser or HN—CH((CH₂)ₙ—N(R⁴R⁵))—C(O);
A⁴ is Lys or HN—CH((CH₂)ₙ—N(R⁴R⁵))—C(O);
A⁵ is Pro;
A⁶ is Asp or HN—CH((CH₂)ₙ—N(R⁴R⁵))—C(O);
A⁷ is Asn or HN—CH((CH₂)ₙ—N(R⁴R⁵))—C(O);
A⁸ is Pro;
A⁹ is Gly or HN—CH((CH₂)ₙ—N(R⁴R⁵))—C(O);
A¹⁰ is Glu or HN—CH((CH₂)ₙ—N(R⁴R⁵))—C(O);
A¹¹ is Asp or HN—CH((CH₂)ₙ—N(R⁴R⁵))—C(O);
A¹² is Ala or HN—CH((CH₂)ₙ—N(R⁴R⁵))—C(O);
A¹³ is Pro;
A¹⁴ is Ala or HN—CH((CH₂)ₙ—N(R⁴R⁵))—C(O);
A¹⁵ is Glu or HN—CH((CH₂)ₙ—N(R⁴R⁵))—C(O);
A¹⁶ is Asp or HN—CH((CH₂)ₙ—N(R⁴R⁵))—C(O);
A¹⁷ is Met or HN—CH((CH₂)ₙ—N(R⁴R⁵))—C(O);
A¹⁸ is Ala or HN—CH((CH₂)ₙ—N(R⁴R⁵))—C(O);
A¹⁹ is Arg or HN—CH((CH₂)ₙ—N(R⁴R⁵))—C(O);
A²⁰ is Tyr or HN—CH((CH₂)ₙ—N(R⁴R⁵))—C(O);
A²¹ Tyr or HN—CH((CH₂)ₙ—N(R⁴R⁵))—C(O);
A²² is Ser or HN—CH((CH₂)ₙ—N(R⁴R⁵))—C(O);
A²³ is Ala or HN—CH((CH₂)ₙ—N(R⁴R⁵))—C(O);
A²⁴ is Leu or HN—CH((CH₂)ₙ—N(R⁴R⁵))—C(O);
A²⁵ is Arg or HN—CH((CH₂)ₙ—N(R⁴R⁵))—C(O);
A²⁶ is H is or HN—CH((CH₂)ₙ—N(R⁴R⁵))—C(O);
A²⁷ is Tyr or HN—CH((CH₂)ₙ—N(R⁴R⁵))—C(O);
A²⁸ is Ile or HN—CH((CH₂)ₙ—N(R⁴R⁵))—C(O);
A²⁹ is Asn or HN—CH((CH₂)ₙ—N(R⁴R⁵))—C(O);
A³⁰ is Leu or HN—CH((CH₂)ₙ—N(R⁴R⁵))—C(O);
A³¹ is Ile, Leu, or HN—CH((CH₂)ₙ—N(R⁴R⁵))—C(O);
A³² is Thr or HN—CH((CH₂)ₙ—N(R⁴R⁵))—C(O);
A³³ is Arg or HN—CH((CH₂)ₙ—N(R⁴R⁵))—C(O);
A³⁵ is Arg or HN—CH((CH₂)ₙ—N(R⁴R⁵))—C(O);
A³⁶ is Tyr or HN—CH((CH₂)ₙ—N(R⁴R⁵))—C(O);
A³⁷ is HN—CH((CH₂)ₙ—N(R⁴R⁵))—C(O) or deleted;
R¹ is NH₂;
R² and R³ each is, independently for each occurrence, H or (C₁₋₃₀)acyl;
provided that when R² is (C₁₋₃₀)acyl, R³ is H;
R⁴ and R⁵ each is, independently for each occurrence, H or (C₁₋₄₀)acyl;
n is 4; and
X¹, X², X³, X⁴, and X⁵ each is, independently for each occurrence, H, CH₂NH₂, or NH₂;
or a pharmaceutically acceptable salt thereof.

3. A compound according to claim 2, wherein HN—CH((CH₂)ₙ—N(R⁴R⁵))—C(O) is Lys(N^{ε}—C(O)—(CH₂)₁₂—CH₃), or a pharmaceutically acceptable salt thereof.

4. A compound according to claim 3, wherein said compound is:
[Leu³¹, Pro³⁴, Lys³⁶(N^{ε}—C(O)—(CH₂)₁₂—CH₃)]hNPY(1-36)-NH₂ (SEQ ID NO:3);
[Leu³¹, Pro³⁴, Lys³⁵(N^{ε}—C(O)—(CH₂)₁₂—CH₃)]hNPY(1-36)-NH₂ (SEQ ID NO:4);
[Lys²⁴(N^{ε}—C(O)—(CH₂)₁₂—CH₃), Leu³¹, Pro³⁴]hNPY(1-36)-NH₂ (SEQ ID NO:5);
[Lys²³(N^{ε}—C(O)—(CH₂)₁₂—CH₃), Leu³¹, Pro³⁴]hNPY(1-36)-NH₂ (SEQ ID NO:6);
[Lys²²(N^{ε}—C(O)—(CH₂)₁₂—CH₃), Leu³¹, Pro³⁴]hNPY(1-36)-NH₂ (SEQ ID NO:7);
[Lys²¹(N^{ε}—C(O)—(CH₂)₁₂—CH₃), Leu³¹, Pro³⁴]hNPY(1-36)-NH₂ (SEQ ID NO:8);
[Lys²⁰(N^{ε}—C(O)—(CH₂)₁₂—CH₃), Leu³¹, Pro³⁴]hNPY(1-36)-NH₂ (SEQ ID NO:9);
[Lys¹⁹(N^{ε}—C(O)—(CH₂)₁₂—CH₃), Leu³¹, Pro³⁴]hNPY(1-36)-NH₂ (SEQ ID NO:10);
[Lys¹⁸(N^{ε}—C(O)—(CH₂)₁₂—CH₃), Leu³¹, Pro³⁴]hNPY(1-36)-NH₂ (SEQ ID NO:11);
[Lys¹⁷(N^{ε}—C(O)—(CH₂)₁₂—CH₃), Leu³¹, Pro³⁴]hNPY(1-36)-NH₂ (SEQ ID NO:12);
[Lys¹⁶(N^{ε}—C(O)—(CH₂)₁₂—CH₃), Leu³¹, Pro³⁴]hNPY(1-36)-NH₂ (SEQ ID NO:13);
[Lys¹⁵(N^{ε}—C(O)—(CH₂)₁₂—CH₃), Leu³¹, Pro³⁴]hNPY(1-36)-NH₂ (SEQ ID NO:14);
[Lys¹⁴(N^{ε}—C(O)—(CH₂)₁₂—CH₃), Leu³¹, Pro³⁴]hNPY(1-36)-NH₂ (SEQ ID NO:15);
[Lys¹²(N^{ε}—C(O)—(CH₂)₁₂—CH₃), Leu³¹, Pro³⁴]hNPY(1-36)-NH₂ (SEQ ID NO:16);
[Lys¹¹(N^{ε}—C(O)—(CH₂)₁₂—CH₃), Leu³¹, Pro³⁴]hNPY(1-36)-NH₂ (SEQ ID NO:17);
[Lys¹⁰(N^{ε}—C(O)—(CH₂)₁₂—CH₃), Leu³¹, Pro³⁴]hNPY(1-36)-NH₂ (SEQ ID NO:18);
[Lys⁹(N^{ε}—C(O)—(CH₂)₁₂—CH₃), Leu³¹, Pro³⁴]hNPY(1-36)-NH₂ (SEQ ID NO:19);
[Lys⁷(N^{ε}—C(O)—(CH₂)₁₂—CH₃), Leu³¹, Pro³⁴]hNPY(1-36)-NH₂ (SEQ ID NO:20);
[Lys⁶(N^{ε}—C(O)—(CH₂)₁₂—CH₃), Leu³¹, Pro³⁴]hNPY(1-36)-NH₂ (SEQ ID NO:21);
[Lys⁴(N^{ε}—C(O)—(CH₂)₁₂—CH₃), Leu³¹, Pro³⁴]hNPY(1-36)-NH₂ (SEQ ID NO:22);
[Lys³(N^{ε}—C(O)—(CH₂)₁₂—CH₃), Leu³¹, Pro³⁴]hNPY(1-36)-NH₂ (SEQ ID NO:23);
[Lys¹(N^{ε}—C(O)—(CH₂)₁₂—CH₃), Leu³¹, Pro³⁴]hNPY(1-36)-NH₂ (SEQ ID NO:24);
[Leu³¹, Pro³⁴, Lys³⁷(N^{ε}—C(O)—(CH₂)₁₂—CH₃)]hNPY(1-37)-NH₂ (SEQ ID NO:25);
[Leu³¹, Lys³³(N^{ε}—C(O)—(CH₂)₁₂—CH₃), Pro³⁴]hNPY(1-36)-NH₂ (SEQ ID NO:26);
[Leu³¹, Lys³²(N^{ε}—C(O)—(CH₂)₁₂—CH₃), Pro³⁴]hNPY(1-36)-NH₂ (SEQ ID NO:27);
[Lys³¹(N^{ε}—C(O)—(CH₂)₁₂—CH₃), Pro³⁴]hNPY(1-36)-NH₂ (SEQ ID NO:28);
[Lys³⁰(N^{ε}—C(O)—(CH₂)₁₂—CH₃), Leu³¹, Pro³⁴]hNPY(1-36)-NH₂ (SEQ ID NO:29);
[Lys²⁹(N^{ε}—C(O)—(CH₂)₁₂—CH₃), Leu³¹, Pro³⁴]hNPY(1-36)-NH₂ (SEQ ID NO:30);
[Lys²⁸(N^{ε}—C(O)—(CH₂)₁₂—CH₃), Leu³¹, Pro³⁴]hNPY(1-36)-NH₂ (SEQ ID NO:31);
[Lys²⁷(N^{ε}—C(O)—(CH₂)₁₂—CH₃), Leu³¹, Pro³⁴]hNPY(1-36)-NH₂ (SEQ ID NO:32);
[Lys²⁶(N^{ε}—C(O)—(CH₂)₁₂—CH₃), Leu³¹, Pro³⁴]hNPY(1-36)-NH₂ (SEQ ID NO:33);
[Lys²⁵(N^{ε}—C(O)—(CH₂)₁₂—CH₃), Leu³¹, Pro³⁴]hNPY(1-36)-NH₂ (SEQ ID NO:34); or
[CH₃(CH₂)₈(CO)-Tyr¹, Nle¹⁷, Pro³⁴]hNPY(1-36)-NH₂ (SEQ ID NO:37) or a pharmaceutically acceptable salt thereof.

5. A pharmaceutical composition comprising an effective amount of a compound according to claim 1 or a pharmaceutically acceptable salt thereof.

6. A pharmaceutical composition of claim 5, further comprising a pharmaceutically acceptable carrier.

7-9. (canceled)

10. A method for treating a disorder or a disease mediated by neuropeptide Y-receptor binding, comprising administering to a subject in need thereof a therapeutically effective amount of a compound of claim 1.

11. The method according to claim 10, wherein said neuropeptide Y receptor is a NPY-Y1 receptor.

12. The method according to claim 10, wherein said disorder or disease pertains to the heart, blood vessels, renal system, vasospasm, heart failure, shock, cardiac hypertrophy, increased blood pressure, angina, myocardial infarction, sudden cardiac death, arrhythmia, peripheral vascular disease, impaired flow of fluid, abnormal mass transport, or renal failure.

13. The method according to claim 10, wherein said disorder or disease is related to increased sympathetic nerve activity.

14. The method according to claim 10, wherein said disorder or disease is related to the central nervous system, cerebral infarction, neurodegeneration, epilepsy, stroke, cerebral vasospasm, cerebral hemorrhage, depression, anxiety, schizophrenia, or dementia.

15. The method according to claim 10, wherein said disorder or disease is related to pain or nociception.

16. The method according to claim 10, wherein said disorder or disease is related to abnormal gastrointcnstinal gastrointestinal motility and secretion, different forms of ileus, urinary incontinence, or Crohn's disease.

17. The method according to claim 10, wherein said disorder or disease pertains to abnormal drink and food intake disorders, anorexia or metabolic disorders.

18. The method according to claim 10, wherein said disorder or disease is related to sexual dysfunction or reproductive disorders.

19. The method according to claim 10, wherein said disorder or disease is associated with inflammation.

20. The method according to claim 10, wherein said disorder or disease is a respiratory disease, asthma or bronchoconstriction.

21. The method according to claim 10, wherein said disorder or disease is related to abnormal release of leutinizing hormone, growth hormone, insulin, and or prolactin.

22. The method according to claim 11, wherein said condition or disease is a tumor expressing the NPY-Y1 receptor.

23. The method of claim 22, wherein said tumor is breast cancer, ovarian cancer, or glioblastoma.

24. The method according to claim 11, wherein said condition or disease mediated by the NPY-Y1 receptor binding is hypertension.

25. The method according to claim 10, wherein said condition or disease is obesity, hyperphasia, or bulimia.


SEQUENCE LISTING (37 sequences)

SEQ ID NO: 1 - protein, 36 aa, Homo sapiens
  MOD_RES (36)..(36): AMIDATION
YPSKPDNPGEDAPAEDMARYYSALRHYINLITRQRY

SEQ ID NO: 2 - protein, 37 aa, Artificial Sequence
  Generic Formula
XXXXXXXXXXXXXXXXXXXXXXXXXXXXXXXXXPXXX

SEQ ID NO: 3 - protein, 36 aa, Artificial Sequence
  Xaa = Lys(myristoyl)
YPSKPDNPGEDAPAEDMARYYSALRHYINLLTRPRX

SEQ ID NO: 4 - protein, 36 aa, Artificial Sequence
  Xaa = Lys(myristoyl)
YPSKPDNPGEDAPAEDMARYYSALRHYINLLTRPXY

SEQ ID NO: 5 - protein, 36 aa, Artificial Sequence
  Xaa = Lys(myristoyl)
YPSKPDNPGEDAPAEDMARYYSAXRHYINLLTRPRY

SEQ ID NO: 6 - protein, 36 aa, Artificial Sequence
  Xaa = Lys(myristoyl)
YPSKPDNPGEDAPAEDMARYYSXLRHYINLLTRPRY

SEQ ID NO: 7 - protein, 36 aa, Artificial Sequence
  Xaa = Lys(myristoyl)
YPSKPDNPGEDAPAEDMARYYXALRHYINLLTRPRY

SEQ ID NO: 8 - protein, 36 aa, Artificial Sequence
  Xaa = Lys(myristoyl)
YPSKPDNPGEDAPAEDMARYXSALRHYINLLTRPRY

SEQ ID NO: 9 - protein, 36 aa, Artificial Sequence
  Xaa = Lys(myristoyl)
YPSKPDNPGEDAPAEDMARXYSALRHYINLLTRPRY

SEQ ID NO: 10 - protein, 36 aa, Artificial Sequence
  Xaa = Lys(myristoyl)
YPSKPDNPGEDAPAEDMAXYYSALRHYINLLTRPRY

SEQ ID NO: 11 - protein, 36 aa, Artificial Sequence
  Xaa = Lys(myristoyl)
YPSKPDNPGEDAPAEDMXRYYSALRHYINLLTRPRY

SEQ ID NO: 12 - protein, 36 aa, Artificial Sequence
  Xaa = Lys(myristoyl)
YPSKPDNPGEDAPAEDXARYYSALRHYINLLTRPRY

SEQ ID NO: 13 - protein, 36 aa, Artificial Sequence
  Xaa = Lys(myristoyl)
YPSKPDNPGEDAPAEXMARYYSALRHYINLLTRPRY

SEQ ID NO: 14 - protein, 36 aa, Artificial Sequence
  Xaa = Lys(myristoyl)
YPSKPDNPGEDAPAXDMARYYSALRHYINLLTRPRY

SEQ ID NO: 15 - protein, 36 aa, Artificial Sequence
  Xaa = Lys(myristoyl)
YPSKPDNPGEDAPXEDMARYYSALRHYINLLTRPRY

SEQ ID NO: 16 - protein, 36 aa, Artificial Sequence
  Xaa = Lys(myristoyl)
YPSKPDNPGEDXPAEDMARYYSALRHYINLLTRPRY

SEQ ID NO: 17 - protein, 36 aa, Artificial Sequence
  Xaa = Lys(myristoyl)
YPSKPDNPGEXAPAEDMARYYSALRHYINLLTRPRY

SEQ ID NO: 18 - protein, 36 aa, Artificial Sequence
  Xaa = Lys(myristoyl)
YPSKPDNPGXDAPAEDMARYYSALRHYINLLTRPRY

SEQ ID NO: 19 - protein, 36 aa, Artificial Sequence
  Xaa = Lys(myristoyl)
YPSKPDNPXEDAPAEDMARYYSALRHYINLLTRPRY

SEQ ID NO: 20 - protein, 36 aa, Artificial Sequence
  Xaa = Lys(myristoyl)
YPSKPDXPGEDAPAEDMARYYSALRHYINLLTRPRY

SEQ ID NO: 21 - protein, 36 aa, Artificial Sequence
  Xaa = Lys(myristoyl)
YPSKPXNPGEDAPAEDMARYYSALRHYINLLTRPRY

SEQ ID NO: 22 - protein, 36 aa, Artificial Sequence
  Xaa = Lys(myristoyl)
YPSXPDNPGEDAPAEDMARYYSALRHYINLLTRPRY

SEQ ID NO: 23 - protein, 36 aa, Artificial Sequence
  Xaa = Lys(myristoyl)
YPXKPDNPGEDAPAEDMARYYSALRHYINLLTRPRY

SEQ ID NO: 24 - protein, 36 aa, Artificial Sequence
  Xaa = Lys(myristoyl)
XPSKPDNPGEDAPAEDMARYYSALRHYINLLTRPRY

SEQ ID NO: 25 - protein, 37 aa, Artificial Sequence
  Xaa = Lys(myristoyl)
YPSKPDNPGEDAPAEDMARYYSALRHYINLLTRPRYX

SEQ ID NO: 26 - protein, 36 aa, Artificial Sequence
  Xaa = Lys(myristoyl)
YPSKPDNPGEDAPAEDMARYYSALRHYINLLTXPRY

SEQ ID NO: 27 - protein, 36 aa, Artificial Sequence
  Xaa = Lys(myristoyl)
YPSKPDNPGEDAPAEDMARYYSALRHYINLLXRPRY

SEQ ID NO: 28 - protein, 36 aa, Artificial Sequence
  Xaa = Lys(myristoyl)
YPSKPDNPGEDAPAEDMARYYSALRHYINLXTRPRY

SEQ ID NO: 29 - protein, 36 aa, Artificial Sequence
  Xaa = Lys(myristoyl)
YPSKPDNPGEDAPAEDMARYYSALRHYINXLTRPRY

SEQ ID NO: 30 - protein, 36 aa, Artificial Sequence
  Xaa = Lys(myristoyl)
YPSKPDNPGEDAPAEDMARYYSALRHYIXLLTRPRY

SEQ ID NO: 31 - protein, 36 aa, Artificial Sequence
  Xaa = Lys(myristoyl)
YPSKPDNPGEDAPAEDMARYYSALRHYXNLLTRPRY

SEQ ID NO: 32 - protein, 36 aa, Artificial Sequence
  Xaa = Lys(myristoyl)
YPSKPDNPGEDAPAEDMARYYSALRHXINLLTRPRY

SEQ ID NO: 33 - protein, 36 aa, Artificial Sequence
  Xaa = Lys(myristoyl)
YPSKPDNPGEDAPAEDMARYYSALRXYINLLTRPRY

SEQ ID NO: 34 - protein, 36 aa, Artificial Sequence
  Xaa = Lys(myristoyl)
YPSKPDNPGEDAPAEDMARYYSALXHYINLLTRPRY

SEQ ID NO: 35 - protein, 36 aa, Artificial Sequence
  Xaa = Lys(myristoyl)
YPSKPDNPGEDAPAEDMARYYSALRHYINLITRPRY

SEQ ID NO: 36 - protein, 36 aa, Homo sapiens; identical to SEQ ID NO: 1

SEQ ID NO: 37 - protein, 36 aa, Artificial Sequence
  synthetic analog of NPY
XPSKPDNPGEDAPAEDXARYYSALRHYINLITRPRY